(19) United States
(12) Patent Application Publication
(10) Pub. No.: US 2004/0199119 A1
(43) Pub. Date: Oct. 7, 2004

(54) Dilator insertion assistance device

(76) Inventors: James J. Alonzo, Carnegie, PA (US); Robert Freyvogel, Butler, PA (US)

Correspondence Address: CARL A. RONALD, ESQ., 1500 ONE PPG PLACE, PITTSBURG, PA, 15222, US

(21) Appl. No.: 10/407,679

(22) Filed: Apr. 4, 2003

(51) Int. Cl. A61M 5/178; A61M 5/00

(52) U.S. Cl. 604/164.1; 604/264

(57) ABSTRACT

An apparatus is disclosed to assist surgeons in performing laparoscopic surgery. The apparatus facilitates insertion of a dilator or trocar into the abdominal cavity of a patient through a much smaller diameter portal created by a Verres needle. A portion of the apparatus of the instant invention attaches to a protruding Verres needle sheath once the needle is removed after insufflation of the patient. A number of different dilator advancement mechanisms are disclosed such as ratchet, screw, pneumatic and hydraulic means that controllably facilitate the insertion of the dilator or trocar.

### CROSS REFERENCE TO RELATED APPLICATIONS

Not Applicable.

### BACKGROUND

Field of the Invention

This invention relates to the use of Verres needles, blunt trocars and dilators in laparoscopic surgery for penetrating the abdomen and particularly to an apparatus for facilitating the insertion of a blunt trocar or dilator into an existing portal of much smaller diameter created by a Verres needle. More specifically, the apparatus of the instant invention concerns a device designed to, among other benefits, drastically reduce the force necessary to insert a dilator or blunt trocar into a patient in the process of performing laparoscopic surgery. Correspondingly, it is anticipated that the instant invention will reduce the trauma to a patient attendant to the surgical procedure, as well as diminishing the possibility for additional unintended injury.

Generally, laparoscopic surgery requires the insertion of some type of hollow tube into the abdominal cavity of a patient to facilitate access to the surgical site. One of several methods by which this is accomplished is the subject of the instant invention. The procedure that is improved by the instant invention starts with the insertion of a specialized needle, called a Verres needle, into the abdominal cavity. This needle is designed to enable gas to be pumped into and retained within the abdominal cavity. This gas pushes tissues and organs away from each other and permits visualization of the diseased organ via a camera and, subsequently, manipulation of surgical instruments inside the patient.

After insufflation of the abdominal cavity is complete and positive pressure is maintained, a trocar is inserted into the patient to create a larger portal for the laparascope. Depending on the preference of the surgeon and the method used, this trocar can either be sharp or blunt. And depending upon the type of equipment chosen and the surgical method employed, the trocar may be inserted via the creation of a separate entry point from the Verres needle or it may utilize the pre-existing portal created by the needle. The instant invention capitalizes on the method that uses the pre-existing Verres needle access point.

It should be noted that insertion of both the Verres needle and the trocar are blind in that the surgeon does not yet have the ability to determine the exact location of certain important vessels and organs within the patient as he or she inserts this equipment. Thus, precision in positioning and delicacy of touch with these tools are paramount. With a sharp trocar, there is the possibility of cutting into a major vessel or organ. A further problem with sharp trocars is that they are generally inserted at a different point than the Verres needle and additional trauma is done to the patient on top of the possibility of slicing the wrong part of the patient.

Blunt trocars or dilators, on the other hand, suffer from the drawback that they require a large amount of force to insert. A surgeon inserting a dilator into a portal created by a Verres needle will need to get a good amount of leverage and use brute force to insert the instrument. Aside from requiring good muscle tone on the surgeon, this method suffers from the drawback that once the blunt trocar or dilator enters the abdominal cavity, resistance to the surgeon's forceful pushing instantly and dramatically decreases and injury can result from over insertion. Organs or vessels can be punctured because the surgeon is not capable of instantly stopping the pushing motion.

Reported injuries to patients from the process of inserting the needle or the trocar include perforation of an artery or vessel. In fact, trocar perforation of a blood vessel is the second most often reported cause of death during laparoscopic surgery (anesthesia being number one). To further complicate matters, an inherent problem with laparoscopic surgery is that any type of injury to the vessels or tissues is not easily recognizable until the patient is in shock because the surgeon is not able to view the injury site.

Other dangers that arise from the insertion of the trocar include gas embolization, bowel perforation, bile leak and pneumothorax. If a blood vessel is ripped during the insertion of the needle or trocar, the gas that is pumped into the body to expand the cavity can enter the blood stream and subsequently, the heart with fatal consequences. It is also possible for a pneumothorax to occur. In this situation, gas enters the lung lining and causes lung collapse or worse. This could happen via trocar injury to the diaphragm, among other things.

Despite these hazards, laparoscopic surgery has become more and more important to health care administrators and patients alike because reduced additional injury to the patient during the operative procedure means a shortened recovery time. Originally developed as a diagnostic tool by gynecologists, the recent trend has seen laparoscopic surgery applied to everything from the removal of the gall bladder to surgery involving the appendix, colon, stomach, kidney and liver.

As health care costs continue to spiral out of control, private community hospitals have more incentive to perform this type of procedure even though they may not have laparoscopic specialists performing the surgery. It is entirely possible that such a facility may only have had a particular surgeon attend a short seminar on the method. Clearly, that surgeon will not have a wealth of experience in performing the procedure. One study showed that physicians who had completed less than 100 laparoscopic procedures had a complication rate of four times higher than more experienced practitioners.

The disclosure of the instant invention is designed to work with existing products such as disclosed in U.S. Pat. No. 5,183,464 to Dubrul, et. al. which insert dilators into small diameter portals and enlarge the opening to provide access ports for laparoscopic procedures. A problem with this type of mechanism is the force required to insert the trocar. Among the objectives of the present invention are to provide an apparatus for facilitating the controllable insertion of a dilator via an existing aperture or portal created by a Verres needle. The emphasis on the present invention is the reduced muscular effort on the part of the surgeon, combined with increased precision and control over the insertion of a blunt trocar or dilator. This valuable goal has not been adequately addressed in the prior art. It is also desirable for the apparatus to be easily sterilized after use.

In accordance with the invention, an apparatus is provided for easily advancing a dilator or blunt trocar into a patient by movement of the instrument to be inserted relative to a previously inserted dilation sheath. Means is provided for the apparatus of this invention to firmly attach to the handle provided by the dilation sheath that was part of a previously inserted Verres needle instrument such that advancement of the dilator is accomplished without removing the sheath from the patient, thus further providing protection to the surrounding tissues from the forced insertion of the dilator or trocar. It will be apparent to those skilled in the art that there could be several different styles or variations on the shape or type of handle.

It is therefore the purpose of the instant invention to develop a device for controlled placement of a dilator or trocar into the same portal created by the Verres needle. This will reduce injury to the patient and correspondingly reduce recovery time and potential complications. No sharp objects need to be involved.

### BRIEF DESCRIPTION OF THE DRAWINGS

The figures show the object in accordance with the invention in a highly schematic manner and are not to be taken to scale.

FIG. 1 shows a plan view of the device in accordance with the instant invention.

FIG. 2 shows a perspective view of the dilator insertion assistance device shown with one type of Verres needle handle and sheath. The device is attached in accordance with the principles of the instant invention and a dilator is shown in a pre-insertion position.

FIG. 3 shows an enlarged fragmentary sectional view of an advancement controller portion of the apparatus shown in FIG. 1.

FIG. 4 shows a top elevational view of FIG. 1.

FIG. 5 shows a sectional view of the apparatus taken along line 24-24 in FIG. 1 with a dilator in an inserted position within a patient.

FIG. 6 illustrates a fragmentary alternative embodiment of an advancement controller constructed in accordance with the principles of the instant invention and having a cogged belt or chain drive.

FIG. 7 illustrates another fragmentary alternative embodiment of an advancement controller constructed in accordance with the principles of the instant invention and having a geared drive.

### DESCRIPTION OF THE PREFERRED EMBODIMENT

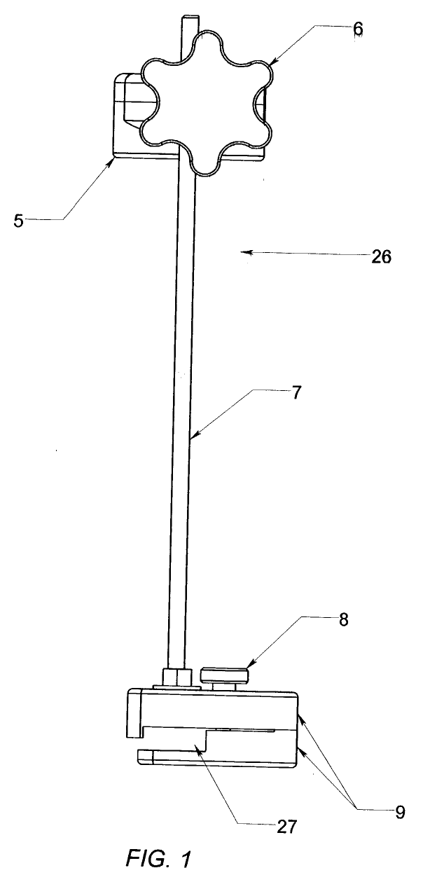

The present invention is a device useful for assisting a surgeon in enlarging an entry point into a patient's abdomen made by a Verres needle through the use of a dilator or trocar. Referring to FIGS. 1-7, several exemplary embodiments of the device of the present invention are shown and are intended for use with a portion of a Verres needle and dilator combination device similar to a device known in the laparoscopic industry as the Innerdyne Tracer Two-Step, that is used in laparoscopic procedures. Again, it will be apparent to those skilled in the art that the device of the instant invention can easily be modified to attach to other Verres needle designs as well.

Throughout the specification, the term “comprising” is used inclusively, in the sense that there may be other features and/or steps included in the invention not expressly defined or comprehended in the features or the steps specifically defined or described. What such other features and/or steps may include will be apparent from the specification read as a whole.

Referring to FIG. 1, the device 26 of the instant invention comprises an insertion means 5 that is manipulated by a user via an insertion means handle 6. When so manipulated, the insertion means cooperatively engages the posts 7 such that the insertion means 5 travels toward the attachment means 9. A slot 27 is created by the surfaces of the attachment means 9 and can be reduced in size in order to be tightened through the use of thumb screws 8 as demonstrated in this embodiment.

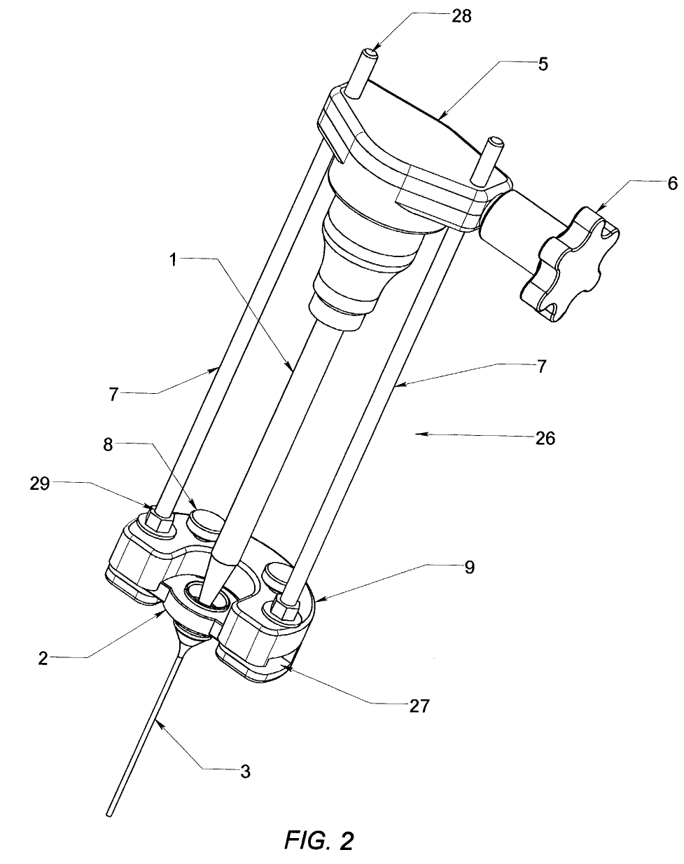

FIG. 2 is a perspective view of the device 26 of the present invention with a mounted dilator 1 axially aligned and ready for insertion through a sheath 3. The attachment means 9 secures the sheath handle 2 in this particular embodiment of the invention through the use of thumb screws 8 that are tightened by a user after the sheath handle 2 is placed in the slot 27 formed by the attachment means 9.

Still referring to FIG. 2, posts 7 extend outwardly from the attachment means 9 and cooperatively engage the insertion means 5 to facilitate advancement of the insertion means 5 from the free end 28 of the posts 7 toward the fixed end 29 of the posts 7 through manipulation of an insertion means handle 6 in accordance with the instant invention.

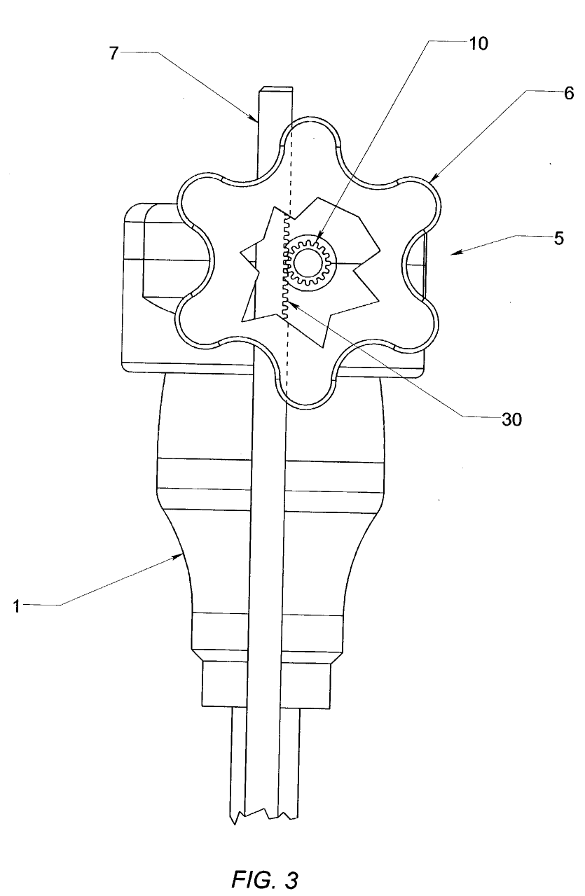

FIG. 3 is an enlarged fragmentary sectional view of on embodiment of an insertion means 5 of the instant invention. It can be easily seen from this view that the insertion means handle 6 is connected to a pinion 10 that cooperatively engages a rack 30 that is affixed or carved into at least one post 7. In this way, the user manipulates the handle 6 to controllably advance the dilator 1 in an intended direction by advancing the pinion 10 along the rack 30.

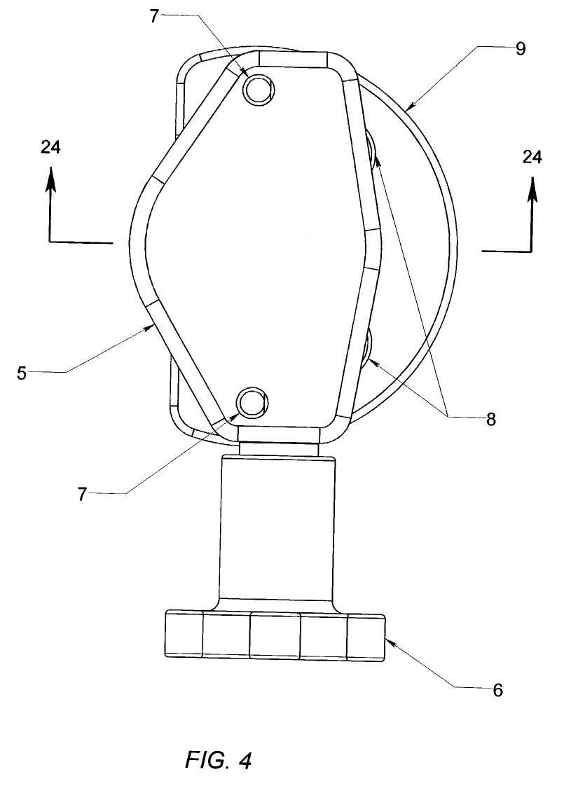

Referring to FIG. 4, the insertion means 5 is prominent in the foreground as is the insertion means handle 6. Manipulation of the insertion means handle 6 causes it to travel along the posts 7 away from the viewer of the illustration toward the attachment means 9. Also evident on this view are thumbscrews 8 that are one embodiment of a clamping device that can be used to tighten the attachment means around the Verres needle handle (not shown) prior to use.

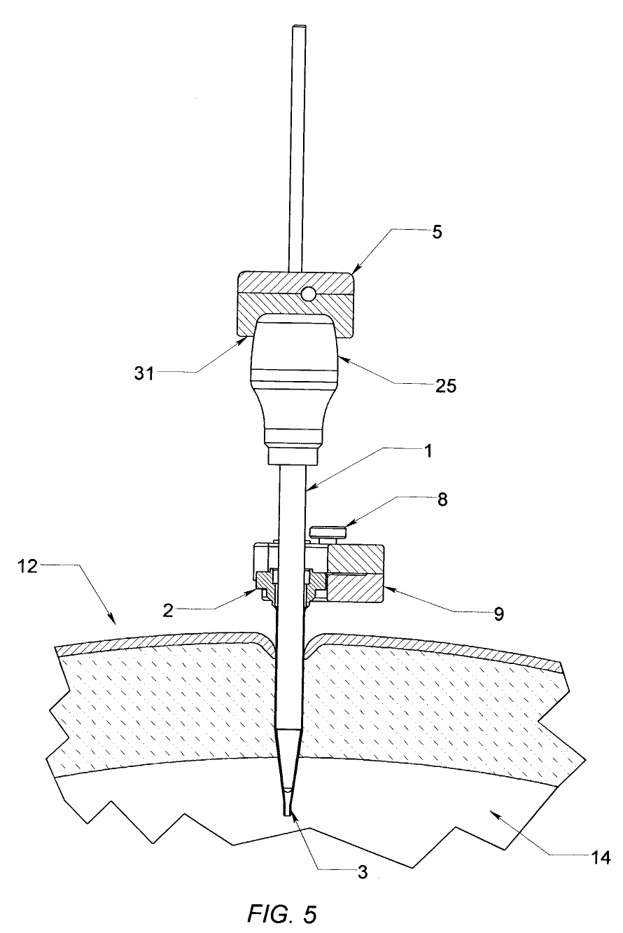

FIG. 5 is a sectional view of the apparatus taken along line 24-24 in FIG. 1 with a dilator 1 in an inserted position within a patient 12. This sectional view demonstrates one embodiment of a dilator handle 25 seated in the insertion means 5. In this embodiment, it can also be seen that the insertion means 5 further comprises a pushing surface 31 that cradles the dilator handle 25.

Still referring to FIG. 5, the dilator 1 has been inserted into the patient 12 and has entered a previously insufflated cavity 14. A portion of the sheath 3 is visible as the dilator 1 has not been fully inserted. The sheath handle 2 has been clamped into the attachment means 9 with the use of a thumb screw 8. While difficult to see in this Figure, the sheath 3 extends along the outside of the dilator 1 from inside the cavity 14 up to the sheath handle 2 as it expands to contain the larger diameter of the dilator 1 while it is being inserted into the patient 12.

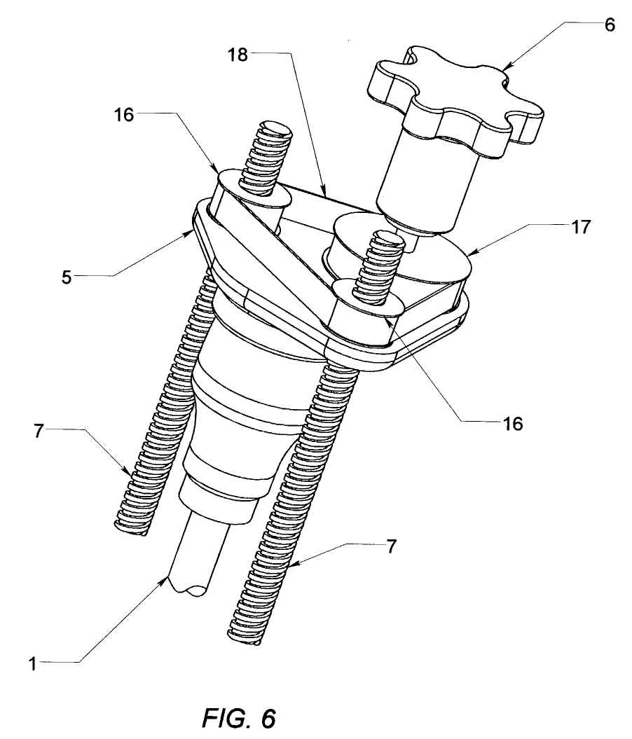

Referring now to FIG. 6, an enlarged fragmentary view of an alternative embodiment of an insertion means 5 is shown constructed in accordance with the principles of the instant invention. The posts 7 are threaded to cooperate with slave sprockets 16 mounted on, so as to be a part of, the insertion means 5. A belt 18 engages the slave sprockets 16 and is moved by a drive sprocket 17. When a user manipulates the insertion means handle 6 that is connected to the drive sprocket 17, the belt 18 moves the slave sprockets 16. Movement of the slave sprockets 16 causes the insertion means 5 to advance along the threaded posts 7 in an intended direction. This advancement, in turn, causes the dilator 1 to be advanced as well.

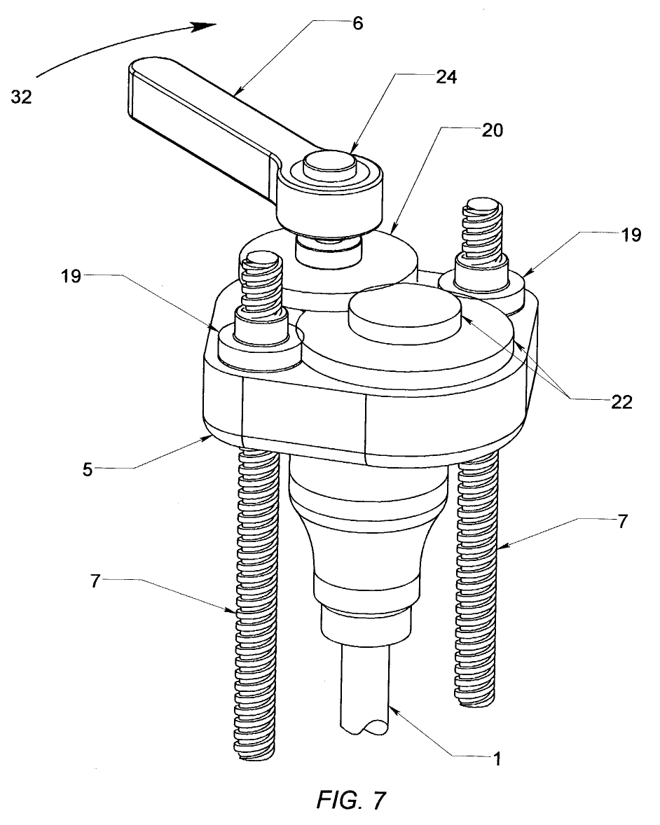

FIG. 7 illustrates an enlarged fragmentary alternative embodiment of an insertion means 5 constructed in accordance with the principles of the instant invention. This is a preferred embodiment due to the fact that a variety of different ratios can be used to achieve an optimum amount of leverage for the user. In this figure, the posts 7 are threaded to facilitate advancement of the insertion means 5 via the slave gears 19. A user manipulates the insertion means handle 6 that works in the same way that a ratchet works i.e. the handle 6 drives the gear shaft 24 when rotated clockwise direction 32 about the gear shaft through an approximately ninety degree angle; however, counter-clockwise rotation to return the handle to its starting position disengages the handle 6 from the gear shaft 24. In this way, the gear shaft 24 rotates the drive gear 20. The drive gear engages a gear set 22 that, in turn, engages the slave gears 19 that, in turn, engage the threaded posts 7 and the dilator 1 is thus advanced.

Since many modifications, variations and changes in detail can be made to the described preferred embodiment of the invention, it is intended that all matters in the foregoing description and shown on the accompanying drawings be interpreted as illustrative and not in a limiting sense. The scope of the invention should be determined by the claims and their legal equivalents. As an example of this principle, the insertion means could be powered by pneumatic or hydraulic means, as well as other equivalent manual types of advancement.

I claim:

1. A dilator insertion assistance device comprising:
insertion means for controllably inserting a dilator into a surgical patient; and
attachment means for fastening the device to a Verres needle sheath; the sheath having been previously inserted into the patient.

2. A dilator insertion assistance device as recited in claim 1, wherein said insertion means further comprises a pushing surface shaped to engage a handled portion of the dilator.

3. A dilator insertion assistance device as recited in claim 2, wherein said attachment means is suitably shaped to receive a Verres needle sheath handle.

4. A dilator insertion assistance device as recited in claim 3, wherein the attachment means further comprises at least one clamping device to firmly grasp the handle.

5. A dilator insertion assistance device as recited in claim 2, wherein the insertion means further comprises at least one post fixably connected to the attachment means at a fixed end; the at least one post extending away from said means and terminating at a free end.

6. A dilator insertion assistance device as recited in claim 5, wherein said attachment means is suitably shaped to receive a Verres needle sheath handle.

7. A dilator insertion assistance device as recited in claim 6, wherein the attachment means further comprises at least one clamping device to firmly grasp the handle.

8. A dilator insertion assistance device as recited in claim 5, wherein the insertion means further comprises a drive mechanism cooperatively engaged with the at least one post to enable controlled advancement of the pushing surface.

9. A dilator insertion assistance device as recited in claim 8, wherein said attachment means is suitably shaped to receive a Verres needle sheath handle.

10. A dilator insertion assistance device as recited in claim 9, wherein the attachment means further comprises at least one clamping device to firmly grasp the handle

11. A dilator insertion assistance device as recited in claim 8, wherein the drive mechanism further comprises a handle means structured to be grasped by a user to facilitate controlled movement of the pushing surface.

12. A dilator insertion assistance device as recited in claim 11, wherein said attachment means is suitably shaped to receive a Verres needle sheath handle.

13. A dilator insertion assistance device as recited in claim 12, wherein the attachment means further comprises at least one clamping device to firmly grasp the handle.

14. A dilator insertion assistance device as recited in claim 1, wherein the insertion means further comprises at least one post fixably connected to the attachment means at a fixed end; the at least one post extending away from said means and terminating at a free end.

15. A dilator insertion device as recited in claim 14, wherein said attachment means is suitably shaped to receive a Verres needle sheath handle.

16. A dilator insertion assistance device as recited in claim 15, wherein the attachment means further comprises at least one clamping device to firmly grasp the handle.

17. A dilator insertion assistance device as recited in claim 14, wherein the insertion means further comprises a drive mechanism cooperatively engaged with the at least one post to enable controlled advancement of the dilator into the sheath.

18. A dilator insertion assistance device as recited in claim 17, wherein said attachment means is suitably shaped to receive a Verres needle sheath handle.

19. A dilator insertion assistance device as recited in claim 18, wherein the attachment means further comprises at least one clamping device to firmly grasp the handle.

20. A dilator insertion assistance device as recited in claim 17, wherein the drive mechanism further comprises a handle means structured to be grasped by a user to facilitate controlled advancement of the dilator into the sheath.

21. A dilator insertion assistance device as recited in claim 20, wherein said attachment means is suitably shaped to receive a Verres needle sheath handle.

22. A dilator insertion assistance device as recited in claim 21, wherein the attachment means further comprises at least one clamping device to firmly grasp the handle.

23. A dilator insertion assistance device as recited in claim 1, wherein said attachment means is suitably shaped to receive a Verres needle sheath handle.

24. A dilator insertion assistance device as recited in claim 23, wherein the attachment means further comprises at least one clamping device to firmly grasp the handle.

25. A dilator insertion assistance device as recited in claim 1, wherein said attachment means further comprises a slot suitably shaped to receive a Verres needle sheath handle.

26. A dilator insertion assistance device as recited in claim 25, wherein said attachment means further comprises at least one clamping device to firmly grasp the handle within the slot.

27. A dilator insertion assistance device as recited in claim 1, wherein said attachment means further comprises at least one clamping device to firmly grasp a Verres needle sheath handle.

28. A method of assisting a surgeon insert a dilator into a laparoscopic patient to enlarge an existing portal created by a Verres needle comprising:
affixing a dilator insertion assistance device to a protruding handle of the a previously inserted needle sheath; and
controllably manipulating an insertion means to gradually insert the dilator into the patient through the sheath.
